(19) United States
(12) Patent Application Publication
(10) Pub. No.: US 2017/0245889 A1
(43) Pub. Date: Aug. 31, 2017

(54) MOVEMENT DAMPENING SYSTEM FOR A SURGICAL TOOL

(71) Applicant: Vanderbilt University, Nahville, TN (US)

(72) Inventors: Stanley Duke Herrell, Nashville, TN (US); Robert J. Webster, Nashville, TN (US); Ray A. Lathrop, Nashville, TN (US)

(21) Appl. No.: 15/513,311

(22) PCT Filed: Sep. 25, 2015
(86) PCT No.: PCT/US15/52416

§ 371 Date: Mar. 22, 2017

Related U.S. Application Data
(60) Provisional application No. 62/055,824, filed on Sep. 26, 2014.

(51) Int. Cl. A61B 17/34; A61B 90/50; A61B 34/00

(52) U.S. Cl. CPC A61B 17/3423; A61B 34/75; A61B 90/50; A61B 2017/3492

(57) ABSTRACT

A movement dampening system for a surgical tool. The system includes a height-adjustable base positionable adjacent to a surgical site on a patient. An elastic damper is disposed adjacent to the height-adjustable base, A channel extends through the elastic damper and the height-adjustable base to the surgical site. A portion of the channel extending through the elastic damper is sized to receive the surgical tool and to restrict movement of the surgical tool.

### CROSS-REFERENCE TO RELATED APPLICATIONS

This application is a non-provisional of and claims priority to U.S. Provisional Patent Application No. 62/055,824 filed on Sep. 26, 2014, the contents of which are incorporated herein by reference.

### BACKGROUND OF THE INVENTION

Tremors of a surgeon's hands are both natural and, in some cases, an impediment to performing tine motions during surgery. As surgical tools become more complex, they offer the ability tier surgeons to perform surgical procedures with a higher degree of complexity and precision, while also presenting new opportunity for error.

### SUMMARY OF THE INVENTION

Tremors and shakes of the hands are a fact of life for surgeons, especially when fatigue is considered. Traditional rigid laparoscopic tools are partially stabilized by the trocar/port where the tool enters the body. The port limits movements of the tool shaft to pivoting about the port or telescoping or rotating about the tool shaft. Tremors that result in forces which don't align with the four degrees of freedom of the surgical tool are naturally attenuated by the surgical port.

Dexterous tools are being developed that have handles which can move in all six degrees of freedom. In these tools, the natural stability provided by a second support point is lost. If the surgical tool has low friction and resistance to movement then any movement of the surgeon's hands will be translated from the handle to the end effector. A rotation of the surgeon's hand results in a movement of the end effector. A rotational tremor of the hand could result in similar movements of the end effector.

A solution to this issue is to add a damper between the main shaft of the tool and the surgical site. The damper resists quick movements of the tool shaft and thus stabilizes the tool against the tissue supporting the port and trocar.

Another solution, which may be used in combination with the damper, is a trocar having a first mass on a distal end in order to effectively locate a combined center of mass of the trocar and a surgical tool near the surgical site. This allows the tool to remain balanced and gives the surgeon more precise control over the tool.

The present invention relates to surgical tool systems and methods, and more specifically, to movement dampening systems for surgical tools.

In one embodiment, the invention provides a movement dampening system for a surgical tool. The system includes a height-adjustable base positionable adjacent to a surgical site on a patient. An elastic damper is disposed adjacent to the height-adjustable base. A channel extends through the elastic damper and the height-adjustable base to the surgical site. A portion of the channel extending through the elastic damper is sized to receive the surgical tool and to restrict movement of the surgical tool.

In another embodiment, the invention provides a method of using a movement dampening system during laparoscopic surgery. A movement dampening system is positioned on an outer surface of a surgical site. The movement dampening system includes a height-adjustable base position adjacent to the surgical site, an elastic damper positioned above the height-adjustable base, and a channel extending through the elastic damper and the height-adjustable base to the surgical site. A portion of the channel extending through the elastic damper is sized to receive a surgical tool and to restrict movement of the surgical tool. A surgical tool is then inserted through the channel and into the surgical site. A height of the height-adjustable base is adjusted to control a dampening characteristic of the movement dampening system. Increasing the height of the height-adjustable base increases a distance between the elastic damper and the surgical site and also increases a dampening effect of the movement dampening system. Conversely, decreasing the height of the height-adjustable base decreases the distance between the elastic damper and the surgical site and decreases the dampening effect of the movement dampening system.

In yet another embodiment, the invention provides a movement dampening and balancing system for a surgical instrument. The system includes a trocar configured to extend into a surgical site of a patient, which has a first mass and a channel. A movement dampening base is coupled to the trocar and adjacent to an exterior surface of the surgical site of the patient. The surgical instrument is configured to extend through the channel and into a patient in order to allow a surgeon to perform a surgical operation. During this operation, the first mass is configured to counter-balance the surgical instrument to result in a combined center of gravity that is near the surgical site.

Other features and aspects of the invention will become apparent by consideration of the following detailed description and accompanying drawings.

### BRIEF DESCRIPTION OF THE DRAWINGS

FIG. 1 is a perspective view of a laparoscopic surgical tool extending through a section of bodily tissue, according to an embodiment of the invention.

FIG. 2 is a perspective view of a laparoscopic surgical tool including a movement dampening system, according to an embodiment of the invention.

FIG. 3 is an enlarged cross-sectional view of the movement dampening system shown in FIG. 2.

FIG. 4 is a perspective view of a movement dampening system according to an alternate embodiment.

FIG. 5 is a perspective view of a movement dampening system according to yet another embodiment of the invention.

### DETAILED DESCRIPTION

Before any embodiments of the invention are explained in detail, it is to be understood that the invention is not limited in its application to the details of construction and the arrangement of components set forth in the following description or illustrated in the Wowing drawings. The invention is capable of other embodiments and of being practiced or of being carried out in various ways. Also, it is to be understood that the phraseology and terminology used herein is for the purpose of description and should not be regarded as limiting.

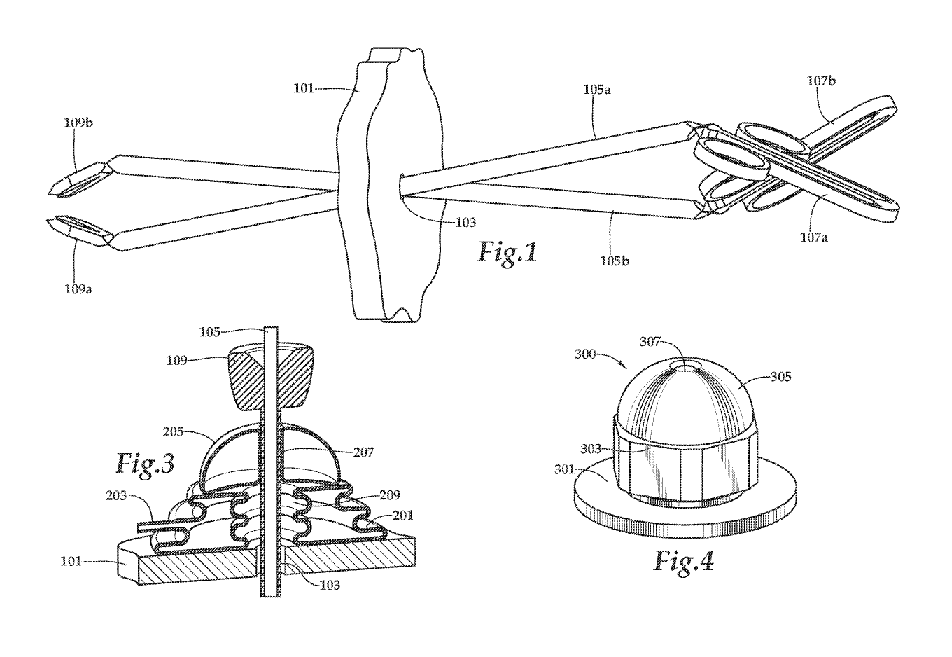

FIG. 1 illustrates a surgical tool, specifically a laparoscopic surgery tool, including a main tool shaft 105A, a handle 107A, and a surgical end effector 109A. The surgical tool is shown inserted through an incision or surgical site 103 in a patient's tissue 101. The position and orientation of the surgical end effector 109A may be displaced or otherwise altered by a user manipulating the handle 107A. FIG. 1 also illustrates the laparoscopic surgical tool in a second position (i.e., main tool shaft 1059, handle 1079, and surgical end effector 109B). Although FIG. 1 illustrates a laparoscopic surgery tool, the inventions described herein are applicable to a wide range of surgical tools (e.g., stereotactic devices, catheters, needles, etc.),

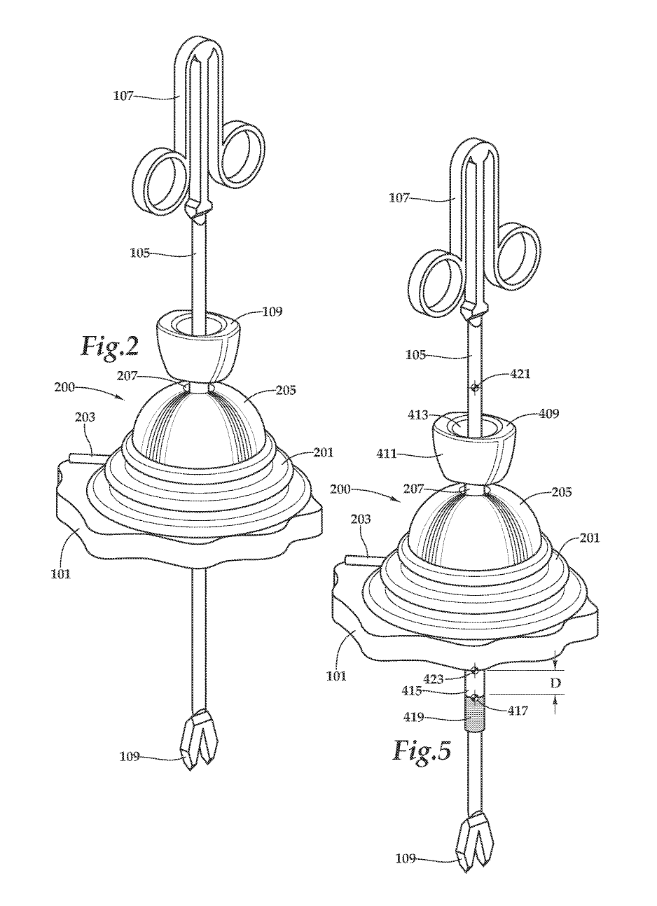

With reference to the embodiment shown in FIG. 2, the surgical tool is used in combination with a trocar 109 and a movement dampening system 200. The movement dampening system 200 includes a height-adjustable base 201, an elastic damper 205, and a channel 207 with an inlet end and an outlet end. An inflation port 203 is used to pump air or fluid into the adjustable height base 201, as described in further detail below. The height-adjustable base 201 defines first surface and a second surface, where the first surface faces the tissue 101 and surgical site 103 of the patient and the second surface faces substantially away from the patient. The elastic damper 205 is coupled to the second surface of the height-adjustable base 201, and defines the inlet end of channel 207. The trocar 109 is inserted into the inlet end of the channel, and extends through the outlet end of the channel thereby entering the tissue at the surgical site. The trocar 109 also defines a cannula that the surgical tool is passed through. However, it should be noted that, in other embodiments (not shown), the surgical tool may be used in conjunction with the movement dampening system 200 in a similar manner, without the use of the trocar 109.

With reference to the embodiment shown in FIG. 3, the interior of the movement dampening system 200 is shown with the trocar 109 and a main shaft 105 of the surgical tool inserted into channel 207. The elastic damper 205 defines a volume, which is filled with a viscous material such as a fluid, gel, grease, or any other suitable material. This material allows for a generally elastic deformation of the elastic damper 205. In other embodiments (not shown), the elastic damper does not define a volume and is instead a suitable elastic material such as rubber. As described above, the elastic damper 205 is coupled to the height-adjustable base 201.

With continued reference to FIG. 3, the height-adjustable base 201 defines an expandable volume in fluid connection with an inflation port 203. The height-adjustable base 201 is constructed from a suitably rigid material that supports and stabilizes the elastic damper 205 with respect to the tissue 101. As shown, the portion of the channel 207 defined by the height-adjustable base has a diameter that is greater than the diameter of the portion of the channel defined by the elastic damper 205. This allows the trocar 109 and the surgical tool to pivot about the surgical site 103. The inflation port 203 allows a medium, such as air or fluid, to be added to the expandable volume, which thus adjusts a height of the base 201 and moves the damper away from the surgical site 103. This increases the damping as it magnifies the displacement of the elastic damper 205 needed for a given movement of the shaft 105 pivoting about the surgical site 103.

With reference to FIG. 4, an alternate embodiment of the movement dampening system 300 is shown. In this embodiment, the movement dampening system 300 includes an elastic damper 305, a base having a seat 301 and an outer member 303, and a channel 307 extending through the movement dampening system 300. The seat 301 defines a lower surface that is configured to abut the tissue 101 of a patient, and further defines a longitudinal axis that is substantially parallel to an axis defined by the channel 307 and substantially perpendicular to a plane defined by the lower surface. The outer member 303 supports the elastic damper 305 and is disposed on an outer surface of the seat 301. The outer member 303 is movably engageable along the longitudinal axis of the seat 301. In this embodiment, the seat 301 is a threaded member, where rotating the outer member 303 displaces the outer member along the longitudinal axis. However, it should be noted that the engageable movement of the outer member may be accomplished by a detent mechanism, a press fitting mechanism or any other combination of devices. Increasing the displacement of the outer member 303 away from the lower surface of the seat 301 increases the damping as it magnifies the displacement of the elastic damper 305 needed for a given movement of the surgical tool in a similar manner as the embodiment described with reference to FIG. 3 above.

In operation, the elastic damper 205,305 is, deflected as the tool pivots about the surgical site. It is noted that surgical tools, such as trocars 109, naturally pivot about the point where they pass through muscular layers near the tissue 101 of the body. Since the pivot point is fixed, the amount of damper deflection is a function of the distance between the damper and the pivot point. Moving the damper further from the pivot, as described in the embodiments above, will effectively increase damping. The height of the height-adjustable base 201,301 can be adjusted before and during a procedure to accommodate the preference of a surgeon.

With reference to FIG. 5, an alternate embodiment of the trocar 409 for use with the movement dampening systems 200, 300 is shown. In the illustrated embodiment, the movement dampening system 200 is shown; however, other systems (i.e., movement dampening system 300) may be used. Alternatively, the trocar 409 may be used without a movement dampening system. The trocar 409 of this embodiment includes proximal end 411 defining a cannula 413 that extends through the trocar 409 to a distal end 415 including a first mass 419. The first mass 419 is generally configured to be disposed at a predetermined depth D within the surgical site, and effectively moves a center of gravity 417 of the trocar 409 closer to the distal end 415.

In operation, the trocar 409 is inserted into the surgical site to in order to locate the first mass 417 at the predetermined depth D. The surgical tool, having a center of gravity 421 near handle 107, may then be passed through cannula 413 in order to perform surgical operations. When the surgical tool is inserted through the cannula 413 of the trocar 409, the first mass 417 acts as a counter balance to orient a combined center of gravity 423 of the surgical tool and the trocar 409 at or near the surgical site (i.e., where the trocar and tool enter the body). This advantageously makes the tool naturally balanced at the surgical site making the tool easier to use. Additionally, a surgeon would not have to exert any extra effort to balance the tool, which reduces surgical errors results in complications and morbidity.

Although the invention has been described in detail with reference to certain preferred embodiments, variations and modifications exist within the scope and spirit of one or more independent aspects of the invention as described.

Various features and advantages of the disclosure are set forth in the following claims.

What is claimed is:

1. A movement dampening system for a surgical tool, the system comprising:
a base positionable adjacent to a surgical site on a patient;
an elastic damper disposed adjacent the base; and
a channel extending through the elastic damper and the base to the surgical site, a portion of the channel extending through the elastic damper being sized to receive the surgical tool and to restrict movement of the surgical tool.

2. The movement dampening system of claim 1, wherein the surgical tool includes a trocar.

3. The movement dampening system of claim 1, wherein the surgical tool includes a laparoscopic device.

4. The movement dampening system of claim 1, wherein elastic damper defines a volume filled with a viscous material selected from a group consisting of a fluid, a gel, and a crease.

5. The movement dampening system of claim 1, wherein the base is a height-adjustable base.

6. The movement dampening system of claim 5, wherein the height-adjustable base includes an expandable volume, and further comprising a pump configured to pump a medium into and out of the expandable volume of the base, wherein pumping the medium into the expandable volume of the base causes the height of the base to increase such that the elastic damper is moved further away from the surgical site.

7. The movement dampening system of claim 5, wherein height-adjustable base includes a seat moveably engageable with an outer member, and wherein a height of the height-adjustable base is adjusted by moving the outer member relative to the seat.

8. The height-adjustable base of claim 7, wherein the seat is threadingly engageable with the outer member.

9. The height-adjustable base of claim 7, wherein the outer member includes a detent spring that engages the seat.

10. The movement dampening system of claim 1, wherein the surgical tool includes a laparoscopic device inserted through a trocar that extends into the surgical site.

11. The movement dampening system of claim 10, wherein the trocar includes a mass disposed on an end configured to be inserted into the surgical site, the mass being configured to counter balance the surgical tool so as to define a combined center of gravity of the surgical tool and trocar near a plane defined by the surgical site.

17. A method for using a movement dampening system during laproscopic surgery, comprising:
positioning a movement dampening system on an outer surface of a surgical site, wherein the movement dampening system includes a height-adjustable base positioned adjacent to the surgical site, an elastic damper positioned above the height-adjustable base, and a channel extending through the elastic damper and the height-adjustable base to the surgical site, a portion of the channel extending through the elastic damper being sized to receive a surgical tool and to restrict movement of the surgical tool;
inserting the surgical tool through the channel and into the surgical site; and
adjusting a height of the height-adjustable base to control dampening characteristics of the movement dampening system,
wherein increasing the height of the height-adjustable base increases a distance between the elastic damper and the surgical site and increases a dampening effect of the movement dampening system, and
wherein decreasing the height of the height-adjustable base decreases the distance between the elastic damper and the surgical site and decreases the dampening effect of the movement dampening system.

13. The method of claim 12, wherein the surgical tool includes a trocar having an opening and first mass disposed on a distal end configured to be located at a predetermined depth within the surgical site.

14. The method of claim 13, further comprising inserting a laparoscopic device through the opening and into the surgical site, such that a combined center of gravity of the laparoscopic device and the trocar is at or substantially adjacent to the surgical site.

15. The method of claim 12, wherein adjusting the height of the height-adjustable base further includes pumping a medium into and out of an expandable volume of the height adjustable base.

16. The method of claim 12, wherein adjusting the height of the height adjustable base includes moving an outer member of the height adjustable base relative to a seat.

17. A stabilizing system for a surgical instrument, comprising:
a trocar, including a first mass and a channel, that is configured to extend into a surgical site of a patient; and
a movement dampening base, disposed adjacent to an exterior surface of the surgical site of the patient, including an elastic member coupled to the trocar;
wherein the surgical instrument is configured to extend through the channel into a patient to perform a surgical operation.

18. The stabilizing system of claim 17, wherein the first mass is configured to counter-balance the surgical instrument to result in a combined center of gravity of both the trocar and the surgical instrument that is near the surgical site.

19. The stabilizing system of claim 17, wherein the movement dampening base includes a height adjustable member supporting the elastic member.

20. The stabilizing system of claim 19, wherein the height adjustable member includes an adjustment mechanism configured to alter a distance between the elastic member and the surgical site to vary an amount of elastic dampening provided by the elastic member.
